(19) United States
(12) Patent Application Publication
(10) Pub. No.: US 2008/0140026 A1
(43) Pub. Date: Jun. 12, 2008

(54) Acoustically-Aided and/or gel-aided delivery of beneficial gaseous, ionic, unstable, metastable, short-lived or reactive species for medical purposes

(76) Inventors: John W. Sliwa, Los Altos, CA (US); Herbert L. Berman, Los Altos Hills, CA (US); Bryan T. Oronsky, Los Altos, CA (US); Carol A. Tosaya, Los Altos Hills, CA (US)

Correspondence Address: David W. Collins;Intellectual Property Law, Suite 100, 512 E. Whitehouse Canyon Road, Green Valley, AR, 85614, US

(21) Appl. No.: 12/001,701

(22) Filed: Dec. 11, 2007

Related U.S. Application Data
(60) Provisional application No. 60/874,689, filed on Dec. 11, 2006.

(51) Int. Cl. A61M 35/00

(52) U.S. Cl. 604/289

(57) ABSTRACT

An apparatus and method are provided for delivering to a patient's first bodily site and allowing bodily transport therefrom to one or more second bodily sites of a medically, physiologically, neurologically or other health-beneficial species, the at least one species or species precursor which would normally be one or more of volatile, gaseous, vaporous, sublimating, reactive, unacceptably degradable, toxic, hazardous, metastable or unstable if stored in unprotected ambient atmospheric conditions

### PRIORITY CLAIM

The present application claims priority from provisional application Ser. No. 60/874,689, filed Dec. 11, 2006.

### BACKGROUND

It is a well known fact that certain gaseous, neutral and/or ionic species are vital to sustain and/or heal the injured or diseased human body. A perfect example of this is oxygen in its various neutral, charged and chemically-combined states. Ambient gaseous molecular oxygen allows for our respiration and oxygen species are increasingly utilized in controlled studies to provide acceleration, if not enablement, of healing processes. Using oxygen-related entities (molecules, atoms, ions, complexes, compounds) as our first teaching example, we demonstrate a method of delivering normally gaseous oxygen entities into the body with high concentration, for example into a wound needing healing, using two different new methods. These methods allow avoidance of the use of current state of the art hyperbaric oxygen treatments which involve the patient visiting a site whereat a hyperbaric pressurization isolation-chamber is entered by the patient for hours at a time on multiple separate occasions. By eliminating the need for the hyperbaric chamber capital-equipment the inventor's apparatus and method makes it possible to treat the human (or animal) body with beneficial gases or gas-sources with a compact device useable anywhere or anytime. Further, the inventive apparatus allows for localized bodily treatment and treatment in which higher fluxes of oxygen-related entities can be delivered. Diabetics, for example, should substantially benefit from the invention in that amputations due to infection may be more often avoided. Additional uses of the invention for enhancing the treatment of cancer and for treating erectile dysfunction are also taught.

### BRIEF DESCRIPTION OF THE FIGURES

FIG. 1 depicts the first (acoustic) approach using an acoustical delivery device treating a wound with one or more oxygen species released or produced at the treatment site from a fluorocarbon streamed parent medium. The acoustical transducer portion might be rigid or flexible. In this example perfluorocarbon molecules also enter the tissue containing or being bound to the oxygen species. However in other cases only the oxygen species or other beneficial species may travel into the tissue.

FIG. 2 depicts the second (gel-based, per our gel definition) approach for treating a wound using a gas-impermeable bandage having a gel source of the beneficial species thereunder. In a first embodiment, passive diffusion of the species takes place into tissue. In a second embodiment, acoustically or electroporation assisted species movement takes place into tissue. As for FIG. 1 in some cases both the species and its parent medium may pass into the tissue.

### DETAILED DESCRIPTION OF THE EMBODIMENTS

### The First Taught Oxygen-Species Embodiment

HBOT (hyperbaric oxygen treatment) is a rapidly growing therapy as evidenced by the rapid growth of facilities having such pressurized chambers and by the increase in the number of clinical studies showing a variety of medical benefits of HBOT for a variety of diseases and injuries. Currently, many such facilities are being constructed-each having one or more hyperbaric chambers capable of treating one or more patients and even up to 10 patients simultaneously. Dr. Paul Harch, Director of the Louisiana School of Medicine, has been quoted as saying “HBOT is going to revolutionize the field of medicine”. Such benefits are seen for pathophysiologic processes and in diseases. HBOT sessions are typically 1-2 hours each and involve entering the claustrophobic chamber after traveling thereto. HBOT is known to stimulate cell growth and repair. For this reason, it has been found beneficial to treat problems related to infections and healing. HBOT is safe and has even been practiced on 6 month old children with pediatric brain injury. Its primary disadvantage is that it is an immobile, expensive and confining piece of capital equipment, requiring the patient to travel to it. A secondary disadvantage is that it can cause barotraumas similar to those experienced when flying, so patients need to swallow often. A third disadvantage is that there is a patient population that should avoid HBOT or at least be watched much more closely during HBOT, such as patients with emphysema, pneumothorax or seizure tendencies.

One of the current leading applications for HBOT is the adjunct treatment of diabetic leg and foot wounds related to insufficient blood flow. The mechanisms of action for HBOT oxygen species are not fully understood and likely include more than just the known physiological process sustainment mechanisms. It is suspected, for example, that HBOT oxygen may one or more of stimulate DNA interactions, act as a messenger stimulating cell repair, or stimulate stem cells. It has been observed that using HBOT even dormant wounds can be encouraged to grow new and healthy tissue. The invention herein encompasses known and future-discovered beneficial effects of oxygen and other species delivered using the invention. It is a tool for delivery of such species, many of which are not currently easily delivered or not deliverable locally.

The healing of diabetic wounds has been a major success for HBOT, but that is not the only known success. Other reimbursable applications of HBOT include treatment of: a) carbon monoxide poisoning, b) cyanide poisoning, c) thermal burns, d) acute or chronic bone infections or osteomyelitis, d) traumatic ischemia, e) crush injuries, and f) necrotizing fascitis.

Two additional applications under investigation by HBOT practitioners include a) children with cerebral palsy and, b) patients with severe traumatic brain injury. Dr Harch believes that cerebral palsy patients improve after HBOT because of increased cell metabolism in damaged brain regions and possibly because of new cell growth. Dedicated HBOT centers for cerebral palsy treatment are even appearing. In the case of traumatic brain injury it has been estimated that death rates might be reduced by as much as 60% using HBOT. Existing and future HBOT applications are prime candidates to be replaced and/or supplemented by the invention herein.

Further out and needing more clinical demonstration are the treatment of acute strokes and spinal cord injuries as well as the treatment of cardiac-surgery patients in order to avoid historic cognitive losses. Note that cognitive losses fall in the category of neurological applications. We include these in our patient-application scope as well.

We define four terms as follows:

### (1) Acoustically-Aided Delivery of Gaseous, Metastable, Unstable or Volatile Species:

We define this to be our inventive delivery of any medically or physiologically beneficial normally-gaseous, volatile, metastable or unstable (in room ambient) species to a treatment site(s) in a living body with the help of acoustics. To be clear, the ultimate internally-beneficial form of the species may be changed from the form it took as it traveled from a source device or reservoir to the treatment site. As an example, molecular O₂ oxygen from an inventive body-external device or reservoir could be acoustically delivered to an at-depth treatment site whereat (or by which time) it is chemically or electrochemically converted to oxygen atoms or ions. Note that this is different from prior art acoustically-aided drug delivery because we are delivering to (or producing at) a treatment site an entity, such as oxygen ions, which are normally gaseous or volatile at ambient conditions. The normally gaseous form (at ambient) is either delivered to the site or is produced at the site by an appropriate precursor (e.g., diatomic O₂ delivery and subsequent disassociation and ionization to ionic oxygen at the site). Thus, our beneficial species will typically be provided by our acoustic injection device as one of i) a gas, neutral, ion or radical in a parent infusate solution or medium (solid, liquid, gel, cream, oil, paste, emulsion, solution, semisolid or suspension parent medium or solution), ii) a cooled or pressurized medium in solid, semisolid, liquid or gel form containing (or being) the species, or iii) a chemical precursor which forms, releases or produces the beneficial gas, ion or radical at the treatment site. So the distinguishing element of this first approach is that the delivery of the species utilizes the help of acoustics such as by providing an added driving force or an increased permeability to the mobile constituents.

### (2) Gel-Sourced Delivery of Gaseous, Metastable, Unstable or Volatile Species:

This second approach does not require but may also use acoustically-aided delivery. For this second approach, a parent gel-like material serves as a source-medium or depot for the beneficial species either in its final employable form or in an intermediate form as for approach (1) above. So the distinguishing feature is a gel-like depot capable of providing the desired beneficial volatile or unstable species. The gel-like depot material may be placed upon or inside the body, invasively, minimally invasively or non-invasively.

In a first example of this a gel, preferably a liquid-form gel that converts to or can be converted to a semi-solid gel at the treatment site, is injected as by hypodermic syringe in liquid or injection-flowable form. The gel contains the beneficial species such as an oxygen species in solution. With time, the beneficial species leaches out of the semi-solidified gel as by diffusion or gel-biodegradation. It may also be released as by chemical or electrochemical interaction with a bodily fluid such as with CSF, blood or urine. A good example would be a liquid-injectable gel that semi-solidifies upon experiencing the 37° C. body heat at the treatment site. The solidified gel acts as a time-release or production depot for the beneficial species such as oxygen ions. Recall again, the actual volatile or unstable species can be formed at the treatment site whereas the delivered (injected) precursor may have a different form on the way to the treatment site.

In a second example of this second approach a gel is placed upon the skin preferably under a barrier-patch or bandage. The preferred patch minimizes the outwards (upwards) passage of the species thereby encouraging it to diffuse-into, pass-into or otherwise be driven-into the tissue. Any provided driving might, for example, include any one or more of iontophoretic mechanisms, electroporation or electrophoretic mechanisms, electroosmotic mechanisms or acoustic mechanisms. Note again that like acoustic mechanisms most of the above electrode-driven mechanisms involve both providing a driving force and providing an easier driving path or increased permeability. So by “driving” we include both providing driving forces and increasing the availability of intracellular or intercellular paths to be driven through. The reader will realize that such driving components could include electrodes and/or ultrasound transducers incorporated into the patch or bandage.

Either the first or second approaches can be utilized to treat superficial tissues or at-depth tissues. In the second approach utilizing a skin-mounted bandage with/without a driving means one can easily and quickly establish a high concentration of oxygen species, for example, in the region of a surface or shallow wound for example. Likewise for an injected solidifying gel (second approach) the gel can quickly locally produce at-depth oxygen species. In the case of an acoustically driven approach (approach 1 if not also approach 2 combined) one can utilize focused or unfocused acoustics to achieve considerable tissue penetration.

So the attentive reader will note that the infusate containing the deliverable beneficial species or its precursor might never itself penetrate the tissue although it may. In the bandage case the infusate may simply serve as the external reservoir for the species and only the species itself diffuses (or is driven) inward and simultaneously out of the infusate parent material. The invention only requires the species or species precursor moves into tissue or body fluid, the parent infusate material holding the species may or may not itself pass into the body in some amount. Finally, the “infusate” might comprise only the species or species precursor without a carrier medium.

### (3) Gel:

Any semisolid or multiphasic substance such as a thick or viscous liquid, paste, cream, foam, network structure, composite material, filled material, emulsion, suspension, thixotropic material or solution that demonstrates some measurable finite resistance to flow or deformation in at least one state. A gel of the invention will typically be gel-like for at least a period. That period could be as-solidified after injection such that it stays in place. It could also be during the period between the patient obtaining a gel-patch and mounting it upon his/her skin, such that during that mounting period the gel does not drip on the floor. Once the patch is mounted, the gel may possibly liquefy. The gel of the invention will preferably be 100% by volume the gel material. However, we include in our scope a “gel” which is a permeable material such as a fabric saturated or wetted with our species or species precursor or species and its parent medium. In this case the permeable material provides some retainment action of the species/medium and the species/medium can therefore, in this special case, be thought of as a “gel” whose viscosity or flow-resistance is increased by the surrounding permeated or soaked fabric.

### (4) Infusible Medium and Beneficial Species:

The beneficial species is what is delivered-to or produced-at the actual treatment site. It may be the beneficial species itself already in useable form or a precursor-form thereof. Recall that the infusible medium may comprise a parent-medium containing the beneficial species in some form or may comprise the beneficial species itself alone-in useable or precursor form. The infusible medium parent material, if any, such as a perfluorocarbon-containing gel with a very high oxygen species solubility, may or may not itself pass into the tissue to be treated. The infusible medium may also contain other drugs or medicaments that pass into the tissue or patient in any manner. The infusible medium itself may be the beneficial species or may be a drug or medicament different than the beneficial species which is also passed into the tissue. The infusible medium and/or beneficial species may be selected to have other desirable properties such as non-toxicity to healthy tissue, electrical conductivity (such as to support electrophoretic or iontophoretic action), photo-optical stability, adhesive properties, flow-resistant properties, antibacterial or antiviral properties, anti-fungal properties or anesthetic properties). So what is to be noted here is that the infusible medium will pass or be passed, at least in part, into or onto tissue which is to benefit from one or more beneficial species in or comprising the infusible medium. The beneficial species includes at least one gaseous, ionic, vaporous, unstable, metastable, reactive, difficult-to-contain or unstable (attributes the species would have presuming these were alternatively held in ambient or unconstrained conditions) species but may also include one or more drugs or medicaments serving the same or other purposes. The inventive species-delivery devices of the invention prevent the beneficial species from diffusing-away, evaporating, otherwise degrading or chemically/electrochemically reacting before it can be delivered to the site of interest. So the device can be viewed as a means to preserve and protect the species until it gets where it is going. Such protection will frequently involve physical containment with regards to evaporation, diffusive-leakage away from the patient, UV degradation protection or reaction avoidance which leads to loss of useable species deliverable to the target site(s). Frequently, the inventive device will provide the beneficial species in a higher concentration than conventional methods. A specific example is oxygen in a perfluorocarbon liquid or gel-such perfluorocarbons can hold large amounts of oxygen in comparison to the concentrations found in the ambient air or even high-pressure oxygen ambients. Thus, the invention might supplant much use of hyperbaric oxygen treatments.

We stress that in combination with the first or second approaches, one might utilize prior-art techniques such as electroporation, iontophoresis or physical micropuncture as by using a microneedle array to increase the ingoing flux of a beneficial species-source or a species itself to or across a treatment site. As an example for the second technique, one could have the above-described oxygen-species delivering bandage also bear an electroporating and/or electrophoretic electrode which drives or increases the tissue permeability of the species.

Key to making the invention practical is the design of the media, medium or infusate which provides the reservoir of beneficial species in usefully high concentration. The present inventors realized that, for example, in the case of oxygen, one can place in solution very, very large amounts of oxygen into perfluorocarbons which can be acoustically streamed into the body using ultrasound (with approach 1 or 2) or which could be a constituent of a gel which is injected or topically applied (with approach 2).

In the case of the oxygen being released from its parent perfluorocarbon medium at or near the tissue target, such release processes can be accelerated as with temperature or ultrasound. We include in our inventive scope the release or production of the beneficial species at or near a treatment site(s) regardless of mechanism. Thus, the final species might simply be released or out-diffused or it might be chemically produced at the treatment site or on the way to the treatment site. If it is locally produced at the site, any manner of pre-cursors or prodrugs may be utilized to form or release it. Also included in our scope is the ultrasound-enhanced or thermally-enhanced release of our beneficial species such as by acoustically, thermally, chemically or electrochemically bursting or breaking-down injected microbubbles as by mechanical rupture, chemical reaction or biodegradation. The acoustic-rupture technique has been explored for local liquid drug release, but to our knowledge has never been used to deliver medically beneficial gases or volatiles other than for acting as a passive gaseous echogenic ultrasound contrast agent. Gel related materials are capable of having very high solubilities for various species such as anticancer drugs. We are adding our species to this list as well as adding the use of ultrasound or temperature to help drive or permeate any of these materials into or through tissues. We also include the use of electric field or electric current technique such as electroporation or iontophoresis to drive a species or help permeate tissue with a species.

Included in our scope of beneficial species are also proteins, enzymes, stem cells, nanoparticles and genetic constituents as well as growth factors. Each of these requires a reservoir design which suppresses the natural evaporation, breakdown or reaction of the species so it is around long enough to be passed into the body from the storage reservoir or depot or our apparatus.

### Discussion of the Figures:

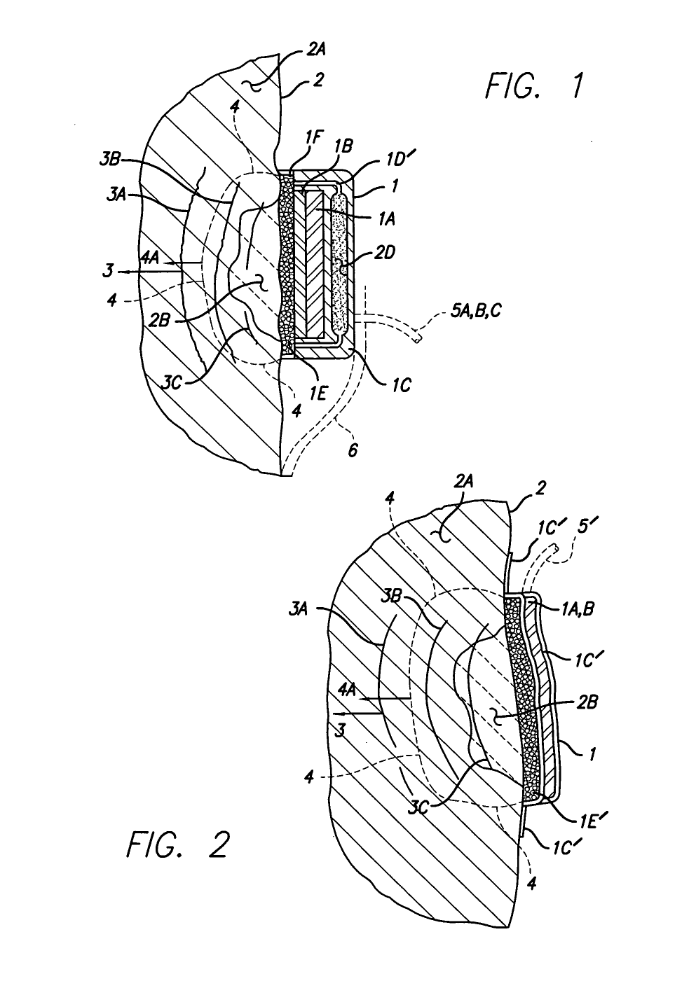

Looking at FIG. 1, we see a depiction of the first approach, that of acoustically aided injection or infusion of an oxygen-species medium such as an oxygen enriched fluorocarbon liquid. Item 1 is our inventive acoustic application device. Inside device 1 we see an acoustic transducer comprising a piezomaterial layer 1a and an optional acoustic matching layer material 1b. Not shown are the required interfacial electrodes and leads to activate the transducer 1a/1b. The device 1 is shown as having an outer body or case 1c and a reservoir 1d for the oxygen or oxygen-based precursor liquid, gel or species alone being infused. Leftwards of the transducer 1a/1b is an intervening permeable layer 1e. Its purpose is to assure that the infusible medium is available between the transducer 1a/1b and the patients skin 2 for infusion by the leftwards progressing acoustic streaming forces. The patient's skin is depicted as item 2 and it will be noted that device 1 is shown situated upon the skin 2 and that device 1 has elastomeric seals if around its edges to minimize leakage or evaporation of the infusible medium laterally away from the device on the skin surface and into the ambient. The infusible medium, e.g., oxygen-bearing fluorocarbon liquid, flows from the reservoir 1d into the permeable layer 1e such as through a conduit 1d′. The patient's skin surface 2 is shown having healthy underlying tissue 2a and a localized injured or infected treatable region 2b situated generally in front of or in transport-communication with the transducer. The transducer is depicted emitting acoustical waves 3 leftwards such as waves 3c, 3b and 3a. These acoustic waves of the type 3a-3c create a leftwards acoustic radiation or streaming pressure which is able to drive the oxygen-bearing medium left-wards as a flux of said medium 4a generally in the direction of the acoustic streaming 3 and toward and into the wound region 2b. Note the phantom outlined region 4. This is the region acoustically infused with the beneficial medium from reservoir 1d after a period of said infusion. It will be noted that the infused region 4 has been formed to include the infected or injured tissue region 2b such that it can be primarily treated. Further items of interest in FIG. 1 shown in phantom include a power and/or electrical cable or umbilical 5a, 5b, 5c which might provide any or all of power, infusion medium or vacuum-clamping for example. Also in phantom can be seen a retaining strap, clamp or bandage 6 which serves to hold the device 1 in place. We explicitly note that the acoustical energy 3 also can serve to increase the permeability or porosity of the tissues as by sonoporation. By “tissues” we mean either injured tissues or healthy tissues through which infusate needs to travel. The device 1 would operate in a pulsed or continuous mode and could also be responsive to feedback from a sensor such as a species sensor. Such a sensor might be invasive or noninvasive and could possibly be a noninvasive optical sensor in the face of the device 1 that measures a parameter of interest such as a tissue species concentration or a tissue (or device) temperature. We have shown a conventional piezoceramic transducer having an acoustic matching layer. It will be appreciated that the transduction means could also comprise a piezopolymer device such as one made of PVDF and therefore being flexible if desired.

Looking now at FIG. 2, we see a second inventive device of the invention depicting the gel-based second approach. This device is a bandage-like entity that infuses our beneficial species or medium plus species into the tissue but preferably does so without the help of acoustics. So, looking at FIG. 2, we see our bandage-like device 1, again mounted on the patient's skin 2. Again, we have a permeable or porous layer 1e′ through which or from which the beneficial medium is passed into the tissue 2, particularly the injured tissue region 2b. We explicitly note in FIG. 2 the impermeable overlayer 1c′ which is impermeable to the species if not also the medium containing it-if any. This sealing layer is a diffusion barrier and combined skin-adhesive means that assures that the beneficial medium does not leak, diffuse away from or evaporate from the treatment site other than into the tissue region 2b. So in this non-acoustic or passive approach, we would still have naturally diffusing beneficial medium plus species (or species alone) moving with a flux 4a into the tissue.

This device of FIG. 2 is likely a non-rigid shapeable bandage-like or compress-like entity, which is shown as self-sealing to the skin.

In a sort of combined embodiment 1 and embodiment 2, FIG. 2 optionally shows a flexible PVDF or piezopolymeric acoustic transducer 1a/b with the appropriate power or umbilical connections 5′. Unlike that of the depicted FIG. 1 device, this FIG. 2 device can be, if desired, flexible and body-contour fitting because PVDF transducers are flexible. We note that in the FIG. 2 device we may utilize an adhesive to not only seal the device 1 to the tissue surface at edges 1c′ but also to hold the device onto the tissue 2 across its full surface. Said adhesive may be arranged to be permeable to the infusing medium or species. So in this flexible acoustic bandage-like option of FIG. 2 we would still see the depicted acoustic waves 3a, 3b, 3c causing the beneficial streaming of the infusate along with any beneficial tissue-permeability alterations.

It is a well established fact that acoustical energy has some purely acoustic benefits such as improving the results of radiation treatment or causing increased beneficial gene transfection or cellular membrane leakage. We include in our scope all such benefits delivered with our inventive devices wherein the device is also capable of delivering one of the inventive beneficial infusates containing a gaseous or volatile (under ambient conditions) species. Thus one could add other drugs or genetic materials to the infusate as well.

We have depicted the devices of FIGS. 1 and 2 as being surface-mounted entities. We expressly state that the inventive devices may also take other forms including those which penetrate the tissue surface or are invasive in whole, i.e. possibly implanted or even ingested. The injected gel example above is certainly a good example of an “invasive” device of the invention albeit the device therein is a solidified gel deposit and the gel-delivery syringe or catheter is likely removed.

### Additional Anticipated Applications: Cancer and Impotence:

Cancer:

Radiation therapy plays a critical role in the local and regional control of malignant tumors. Its efficacy, however, is limited by a number of factors. For example, it is often not possible to deliver the amount of radiation required for tumor control because of dose-limiting toxicity in normal tissues. Large tumors may also contain a greater proportion of regions of relative hypoxia than smaller tumors, which are more resistant to radiotherapy than well oxygenated regions of tumors. Multiple studies have confirmed the presence of regions of both chronically and transiently hypoxic cells in solid tumors. Tumor vasculature is highly irregular, with tortuous and chaotic vessels, arteriovenous shunts, blind ends and incomplete endothelial linings and frequently lack smooth muscle or nerves. As a result, blood flow is often sluggish and sporadic, making oxygen delivery inefficient. The end result is that solid tumors have regions of relatively low oxygenation (or pO₂) levels and many areas of extremely hypoxic cells. When tumor cells are further from blood vessels than the diffusion distance of oxygen (100 micrometers), they are so-called “chronically” hypoxic. The remaining cells, which are subject to sporadic blood flow from particular vessels, are termed “acutely” hypoxic.

These hypoxic regions are relatively radioresistant. Oxygen is required for the formation of radiation-induced reactive oxygen species (ROS), which mediate DNA and other cellular damage. As the most electron-affinic molecule in the cell, oxygen reacts extremely rapidly with free electrons, thereby “fixing” the radiation damage. In the absence of oxygen, much of the radical damage can be restored to its undamaged form by hydrogen donation from non-protein sulfhydryls in the cells.

One method which may overcome this problem is improved oxygen delivery. This idea is not novel as hyperbaric oxygen has been used in conjunction with radio-therapy and a current compound called RSR13 (Efraproxyn™—efaproxiral) which facilitates the release of oxygen from hemoglobin is currently undergoing testing in clinical trials.

Our novel idea herein involves using ultrasound to infuse the tissues with oxygen, thereby increasing tissue pO₂, which may enhance the effects of radiation therapy by enhancing the production of reactive oxygen species in tumor tissue. We believe ultrasound-infused oxygen can be used in conjunction with radiotherapy or other radiosensitizers in various cancers such as melanomas, head and neck, tumors, localized breast cancers, uterine and cervical cancers and prostate cancer, to name a few. By the same token, our inventive non-ultrasound bandage or patch which out-diffuses oxygen or species may also be utilized. By radiation treatment we include all manner of delivering such radiation including beam-directed and seed-sourced radiation.

Impotence:

One of the main physiological causes of impotence, or erectile dysfunction, is the inability of the blood vessels in the penis to dilate enough to allow blood flow and engorgement. Part of the process of erection involves the release of nitric oxide (NO) in the corpus cavernosum which then activates the enzyme guanylate cyclase. This results in increased levels of cyclic guanosine monophosphate (cGMP), leading to smooth muscle relaxation in the corpus cavernosum, resulting in increased inflow of blood and an erection. Sildenafil® or Viagra® is a potent inhibitor of cGMP specific phosphodiesterase which is responsible for degradation of cGMP in the corpus cavernosum As a result, after taking Viagra®, normal sexual stimulation leads to increased levels of cGMP in the corpus cavernosum, which leads to better erections. Other drugs that operate by the same mechanism include Cialis® and Levitra®.

Amongst Viagra's serious adverse effects are: priapism, severe hypotension, myocardial infarction, ventricular arrhythmias, sudden death, stroke and increased intraocular pressure.

Common side effects include headache, flushing, dyspepsia, prolonged erections, palpitations and photophobia. Visual changes include blurring of vision and a bluish tinge of vision.

In order to avoid these potentially serious and/or troubling systemic side effects associated with the use of cGMP phosphodiesterase inhibitors, we propose to infuse NO directly into the corpus cavernosum via ultrasound streaming, ultrasound sonoporation, electroporation, iontophoresis or natural diffusion to promote erection.

Treatment of Chronic Sinusitis:

Mucous stagnation in the sinus secondary to anatomic variations or mucosal edema arising from various etiologies (e.g., acute viral or allergic rhinitis) forms a rich medium for the growth of various pathogens which, in acute sinusitis, usually involves only one type of aerobic bacteria. With persistence of the infection, anaerobic organisms contribute to the pathogenesis. Most cases of chronic sinusitis develop in patients with acute sinusitis that does not respond to treatment or in those who have not received treatment. In chronic rhinosinusitis refractory to medical therapy or with overlying medical illness such as cystic fibrosis or ciliary dysmotility, endoscopic sinus surgery has become a mainstay of treatment. Other modalities, including sinus aspiration and irrigation, and adenoidectomy, may be used in selected patients instead of surgery. Our idea involves the placement of a “bandage”, “strip” or patch/depot impregnated or infused with oxygen species placed over the face much like a BreatheRight® strip, for example, that could diffuse into the frontal, maxillary or ethmoidal sinuses and make the sinusitis more amenable to antibiotic treatment thereby forestalling surgery by eliminating the anaerobic component of the infection and also by providing the host immune cells with an ample source of oxygen with which to exert their micobicidal activity. A more “energetic” infusion of oxygen as by ultrasound could also serve to “agitate’ the secretions inside the sinuses, thereby eliminating the kind of stasis that predisposes to further infection.

Treatment of Myocardial Infarction (MI), Unstable Angina, Stroke, Transient Ischemic Attacks:

The old adages that “time is muscle” or “time is brain” refer to the urgency of restoring oxygen-rich blood flow to ischemic muscle as in the case of the heart or tissue or as in the case of the brain in order to prevent irreversible cell death. Once perfusion to the heart muscle or brain tissue is compromised as by a clot, a small window of opportunity most likely the first 60 minutes of symptom onset, the so-called “golden hour” in MI, exists to restore the blood flow by removing the occlusion before irreversible necrosis sets in. Current guidelines dictate that maximum benefit is achieved by early thrombolysis either achieved mechanically (as in MI) with percutaneous transluminal coronary angioplasty or through the administration of thrombolytics. However, with increasing duration and severity of ischemia, greater damage can develop through a phenomenon called reperfusion injury. The restored blood flow reintroduces oxygen into a previously ischemic environment which can lead to the formation of oxygen free radicals and further damage the cell membranes.

Our idea involves using ultrasound over the chest wall to “force” oxygen into the cardiac myocardium and cardiac vessels, supersaturating them with oxygen during a cardiac event. If instituted promptly, this would in a sense bypass the obstruction and extend the “window of opportunity” or “golden hour” for the administration of interventional or thrombolytic therapy by providing the ischemic muscle with a source of oxygen.

The same idea may be used in stroke therapy, wherein oxygen may be directly infused to the cerebral tissue either through the skull, the cribiform plate in the nose or into the carotid and/or vertebral-basilar circulations in the neck, presumably downstream from any obstruction. A non-ultrasonic approach may also be used with a likely slower delivery rate or time-delay. Included in the inventive scope is an oxygen (or other species) wrap covering a significant body area, such as an entire limb, the back, or the chest. This could be, for example, our second gel-bandage embodiment incorporating oxygen impregnated perfluorocarbons.

Abscess Treatment:

An abscess is a circumscribed collection of purulent material which can occur anywhere in the body. The blood supply to an abscess is frequently poor so antibiotics cannot reach it, which is why in general it must be opened and drained in order to improve. Furthermore, neutrophils and macrophages, which exert their bactericidal activity through the production of reactive oxygen and nitrogen intermediates, may not have enough oxygen and nitrogen due to the poor vascularization of an abscess and in addition many abscesses are caused or complicated by anaerobic bacteria. Our strategy involves infusing a combination of oxygen and nitric oxide (NO) into an abscess in the hopes of exerting a synergistic effect by exposing the bacteria to levels which may inhibit or kill them, as well as providing sufficient oxygen and nitrogen for white blood cells to exercise their killing power. The use of ultrasound to infuse the gases could also provide a measure of local heating which, like the effects of fever, might serve to activate the metabolism of the white cells and hinder those pathogens with strict temperature preferences. Such a treatment strategy might reduce the intravenous course of antibiotics and, consequently, the duration of hospitalization. We include in the scope heating by any means.

Chronic Prostatitis Treatment:

Anaerobes have been implicated in chronic prostate disease and PID. Chronic prostatitis is traditionally treated with prostatic massage and antibiotics. Often, anaerobes are covered empirically on the assumption that all these bacteria cannot be cultured or are too expensive to culture with current culture methods. Prostatic massage with DRE that also infused oxygen into the tissue on a finger-mounted device may aid the treatment of this pathology and shorten the course of antibiotic treatment.

Periodontal Disease Treatment:

Oxygen-laden strips or gel-moldings may be placed over the gums, because these infections are mostly caused by anaerobes.

What is claimed is:

1. An apparatus for delivering to a patient's first bodily site and allowing bodily transport therefrom to one or more second bodily sites of a medically, physiologically, neurologically or other health-beneficial species, the at least one species or species precursor which would normally be one or more of volatile, gaseous, vaporous, sublimating, reactive, unacceptably degradable, toxic, hazardous, metastable or unstable if stored in unprotected ambient atmospheric conditions comprising:
a protective or stabilizing reservoir capable of at least short term stable or safe storage of the beneficial species or species precursor or of a medium or infusant containing the species or precursor;
at least one such species, species precursor, medium or infusant being placed in said reservoir for use of the apparatus;
the apparatus delivering from the reservoir any one or more of the species, species precursor, or a medium or infusant containing the species upon, on or into a bodily tissue or fluid first site from which at least some species or species precursor can subsequently be transported at least some distance within or into the body to at least a second bodily site away from the first delivery site utilizing at least one transport mechanism; and
delivery to the first site;
at least the species also being protected or stabilized as it passes along any possible intermediate path between the reservoir and the first site,
the apparatus optionally also delivering in any manner to any same or different sites in or on the patient's body a drug, medicament, nutrient or physiologically-required material differing in some respect from the species and which serves any patient-beneficial purpose.

2. The apparatus of claim 1 wherein said at least one transport mechanism is at least one of:
a) diffusion or permeation to, onto, into or through tissue or bodily fluid such as by diffusion or permeation along concentration gradients;
b) mobility or permeation enhancement to, onto, into or through tissue or bodily fluid such as by application of electroporation, sonoporation, osmotic mechanisms, tissue-heating which dilates microcapillaries or blood lumens, drugs, mechanical tissue-disruptors such as microneedle arrays, all of which increase permeability;
c) provision of an applied or assistive diffusion or permeation driving force to, onto, into or through tissue or body fluid such as of acoustic streaming or application of electric fields as for electrophoretic or iontophoretic enhanced transport, all of which drive species; or
d) any transport in the bloodstream or in any other flowable or liquid bodily fluid.

3. The apparatus of claim 1 wherein the delivery to the first site includes any one or more of:
a) contacting the apparatus to a patients bodily tissue or fluid for a short or prolonged period;
b) presenting the apparatus to a bodily tissue or fluid at a standoff distance and spraying, pouring, ejecting, extruding or dripping at least the species upon or into the patients body tissue or fluid;
c) coupling the apparatus to a bodily tissue or fluid with a flowable or injection lumen which allows at least the species to be flow-delivered from the reservoir to, upon or into the patients bodily tissue or fluid in any of a liquid, vaporous or gaseous form;
d) use of the apparatus to physically place on or in the body at least some preformed, metered or controlled amount or volume of at least the species in a solid or substantially unflowable form;
e) inhalation, ingestion or any type of bodily or limb immersion; or
f) the use of tissue disruption or disruptors, including a syringe or microneedle(s).

4. The apparatus of claim 1 wherein any of:
a) the apparatus includes, contains or utilizes a reservoir and an acoustic transducer;
b) the apparatus includes, contains or utilizes a reservoir and one or more electrodes utilized to apply an electric field or current which is employed to one or both of provide a species driving force or to increase the tissue/fluid permeability to the species or species precursor; or
c) the apparatus includes, contains or utilizes a reservoir and a skin puncturing or disruption means, including a microneedle(s) which serves to increase tissue permeability to a species or species precursor.

5. The apparatus of claim 1 wherein any of:
a) a species or species precursor-wetted or permeated member is abutted or juxtaposed to patient tissue or fluid out of or from which species or species precursor is transported to, onto, into or through said tissue or fluid;
b) wherein the wetting or permeating species or species precursor is delivered to the member from the reservoir;
c) wherein the member is also the reservoir or has a member-portion resident in the reservoir; or
d) wherein at least one of the following delivery mechanisms is also utilized:
1) contacting the apparatus to a patients bodily tissue or fluid for a short or prolonged period,
2) presenting the apparatus to a bodily tissue or fluid at a standoff distance and spraying, pouring, ejecting, extruding or dripping at least the species upon or into the patients body tissue or fluid,
3) coupling the apparatus to a bodily tissue or fluid with a flowable or injection lumen which allows at least the species to be flow-delivered from the reservoir to, upon or into the patients bodily tissue or fluid in any of a liquid, vaporous or gaseous form,
4) use of the apparatus to physically place on or in the body at least some preformed, metered or controlled amount or volume of at least the species in a solid or substantially unflowable form,
5) inhalation, ingestion or any type of bodily or limb immersion, or
6) the use of tissue disruption or disruptors, including a syringe or microneedle(s).

6. The apparatus of claim 1 wherein a reservoir or reservoir portion of the apparatus is any of:
a) a pressure-containing component which can maintain at least some pressure or partial-pressure gradient from inside to the outside ambient for at least one constituent therein;
b) confining to a species in any manner which limits the species or species precursor's transport in an undesirable direction, including leaking into the ambient;
c) mounted upon or affixed to the patient's skin or tissue in any manner, directly or indirectly, including by utilizing an adhesive, fasteners, belts, straps, clips, clamps, buckles, suction or tissue-penetrating members including microneedles;
d) relatively rigid in design or flexible in design;
e) has an open face, port(s) or flowline(s) placed against, facing or in transport communication with the patient's tissue or body fluid thereby allowing transport out of said reservoir of at least the species or precursor toward, onto, into or through said tissue and fluid but not appreciably into the ambient;
f) has flowline(s) or port(s) which allow delivery of at least the species or precursor toward, onto, into or through the patients tissue or fluid, including directly to said tissue or indirectly as into an intermediate species-permeated member;
g) placed in a patient's body, including by short-term or long-term implantation; or
h) is rechargeable or refillable with species or species-precursor without removing the patient-coupled apparatus from the patient.

7. The apparatus of claim 1 wherein a gel-like material is employed in or with the apparatus in one or more of the following manners:
a) the gel-like material stores, holds, carries, passes along a path toward the patient, or transports at least a species or species-precursor;
b) the gel-like material contacts a patient tissue or body fluid and serves to at least pass along a path toward the patient or transport a species or species precursor;
c) the gel-like material serves as any part of a reservoir;
d) the gel-like material applies or has applied to, across or from it acoustic energy, heat, an electric field or an electrical current;
e) the gel-like material has or is modified to have a desired electrical or acoustical property;
f) the gel-like material conforms or wets to a patient's tissue or body fluid;
g) a gel-like material constituent is a species or species-precursor;
h) a gel-like material is in the form or a disposable element of the apparatus;
i) a gel-like material transports or provides a drug or medicament different than the species or species precursor and possibly for a different purpose;
j) a gel-like material has a high solubility or affinity for an otherwise volatile gas, including O, O₂ or NO;
k) contains a fluorocarbon or perfluorocarbon; or
l) contains a fluorocarbon or perfluorocarbon that carries or is itself a beneficial species.

8. The apparatus of claim 7 wherein the gel-like material is or is also any one or more of: a) a network gel, b) a hydrogel, c) a polymeric gel, d) a thick liquid, e) a viscous liquid, f) a thixotropic material, g) a cream, h) a paste, i) an emulsion, j) an ointment, k) an adhesive, l) a non-runny suspension or solution, m) a multiphase material, n) a material that undergoes a phase change during application or use, o) an organic or inorganic gel, o) species-rechargeable gel, or p) a gel with a fluorocarbon component.

9. The apparatus of claim 1 wherein the apparatus or reservoir thereof remains any of upon, in, contacting, or in transport communication with the patient's tissue, fluids or body while it delivers its beneficial species or species precursor to the first site or while species is transported to a second site.

10. The apparatus of claim 1 wherein at least a species or species precursor can be replenished in any manner after a period of apparatus use or species or species precursor transport, including any one or more of:
a) recharging of the reservoir with at least the species;
b) recharging of a gel-based reservoir with a new quantity of gel and/or species; or
c) replacement of a used or depleted reservoir with a pre-filled reservoir of any type.

11. The apparatus of claim 1 wherein the beneficial species or species-precursor is any of:
a) delivered to a first site(s) via injection of any type, thereafter allowing said transport to the bodily tissue or fluids second site(s) from the injection first site(s) site, the injector being coupled to a reservoir containing at least said species or precursor;
b) delivered via injection to a first site(s) using a syringe, pump, or other pressure-providing container, the syringe, pump or container acting as or being flow-coupled to a reservoir, thereafter allowing said transport to a patient's second site tissues or fluids, including by diffusion or blood-circulation away from an injection first site, the injection pressure being any desired pressure including negligible pressure or gravity pressure;
c) delivered by pouring, spraying or packing into a wound or body cavity first site(s), thereafter allowing said transport to the second site(s) bodily tissue or fluids from that first site(s), the reservoir being any containerization of the species used during the act of depositing it;
d) delivered to a first site(s) by wiping onto or wetting to a patient tissue or fluid from a reservoir, thereafter allowing for transport thereof to second site(s); or
e) delivered by inhalation or ingestion from a reservoir or tank to a first site(s), including the lungs or stomach, thereafter allowing for further transport away from the inhaling or ingestion cavity to a second site(s), including the bloodstream.

12. The apparatus of claim 1 wherein the beneficial species or a precursor or constituent thereof is or is part of an injected gel-like material, the material being injection-flowable during injection and being substantially reduced in flowability and becoming gel-like after some period in the patient's tissue, fluid or body, said reduced flowability possibly being caused by gelling action triggered by body heat, body chemistry, or artificially provided heating.

13. The apparatus of claim 1 wherein the species or a species precursor any one or more of:
a) is dissolved in a parent medium or infusate at any point;
b) is suspended in a parent medium or infusate at any point;
c) is entrained in a parent medium or infusate at any point;
d) is weakly chemically bound to any portion or constituent of a parent medium or infusate at any point;
e) is known to have a high solubility in a parent medium or infusate;
f) is an oxygen related species;
g) is an NO or other nitrogen-oxygen related species;
h) is a species normally utilized by the body in support of a natural bodily function;
i) is a species capable of diffusing or permeating into tissues or body fluids via its own concentration gradient;
j) is a species capable of diffusing or permeating into tissues or body fluids with the help of an additional driving force provided by the apparatus;
k) is a species capable of diffusing or permeating into tissues or body fluids with the help of an additional permeabilizing means provided by the apparatus;
l) is or becomes an ion or radical for at least a period of time;
m) is or becomes a compound for at least a period of time;
n) changes physical phase for at least a period;
o) undergoes a chemical reaction at any point; or
p) is stored, carried in, or transported through a gel-like material at any point.

14. The apparatus of claim 1 wherein a condition, disease or malady being treated is one or more of:
tissue or blood infection;
wound healing;
poisoning;
burn treatment;
bone infection;
traumatic ischemia;
stroke;
potential or actual cognitive loss due to surgical procedures or stroke;
crush injuries;
sports injuries;
cerebral palsy;
spinal cord injury;
severe traumatic brain injury;
diabetic infection or wound treatment;
organ transplants;
skin grafts;
neural diseases;
immune system diseases;
cancer;
impotence;
chronic sinusitis;
myocardial infarction, unstable angina, stroke, or transient ischemic attacks;
abscess treatment;
chronic prostatitis treatment; or
periodontal disease.

15. The apparatus of claim 1 wherein the apparatus is at least one of is:
useable by a patient;
useable at home;
useable by any one or more of a clinician, technician or nurse;
useable by a doctor;
useable while ambulatory;
useable while in bed;
useable with a species or species precursor which requires a prescription;
useable with only temporary handheld juxtaposition or contact to the tissue or body fluid;
useable with the apparatus being attached to or constrained by a patient tissue or body fluid for at least a period of time;
useable while operating on an internal power source;
useable while operating on an external power source; or
useable without power.

16. The apparatus of claim 1 wherein the apparatus has at least one of:
a totally or partially non-disposable nature;
a totally or partially disposable nature;
a consumed component or material including gel-pads, skin-contacting species-permeable members, electrodes, batteries, adhesive attachment components, or a microneedle or array thereof;
a refillable or exchangeable reservoir;
a tissue or limb attachment means;
an injection or pumping means;
a reservoir or source of infusate or beneficial species or both that is maintained at a controlled pressure or a controlled temperature during device use.

17. The apparatus of claim 1 wherein the infusate medium or any other apparatus portion also carries or provides a medicament, drug or nutrient that addresses the same or a different patient issue or need.

18. The apparatus of claim 1 wherein it is carried or worn by a patient or subject.

19. The apparatus of claim 1 wherein a parameter regarding its operation or state is obtained using a sensor.

20. The apparatus of claim 1 wherein it is worn under clothing.

21. The apparatus of claim 1 wherein it is at least somewhat formable or compliant to a patient's body or tissues.

22. The apparatus of claim 1 wherein it is utilized in a bodily cavity, including an oral, rectal or vaginal cavity.

23. The apparatus of claim 1 wherein it contains a warming or heating means which contributes, directly or indirectly, to either of improved delivery or transport rates of species or species precursor to or into the patient or subject.

24. The apparatus of claim 1 wherein it replaces at least some hyperbaric oxygen chamber use.

25. The apparatus of claim 1 wherein it replaces an alternative method of species inhalation or ingestion.

26. A method for delivering to a patient's first bodily site and allowing bodily transport therefrom to one or more second bodily sites of a medically, physiologically, neurologically or other health-beneficial species, the at least one species or species precursor which would normally be one or more of volatile, gaseous, vaporous, sublimating, reactive, unacceptably degradable, toxic, hazardous, metastable or unstable if stored in unprotected ambient atmospheric conditions comprising:
providing a protective or stabilizing reservoir capable of at least short term stable or safe storage of the beneficial species or species precursor or of a medium or infusant containing the species or precursor;
placing at least one such species, species precursor, medium or infusant in said reservoir for use of the apparatus; and
situating the apparatus to deliver from the reservoir any one or more of the species, species precursor, or a medium or infusant containing the species to, upon, on or into a bodily tissue or fluid first site from which at least some species or species precursor can subsequently be transported at least some distance within or into the body to at least a second bodily site away from the first delivery site utilizing at least one transport mechanism,
wherein at least the species is also protected or stabilized as it passes along any possible intermediate path between the reservoir and the first site, and
wherein the apparatus optionally also delivers in any manner to any same or different sites in or on the patient's body a drug, medicament, nutrient or physiologically-required material differing in some respect from the species and which serves any patient-beneficial purpose.

27. The method of claim 26 wherein said transport mechanism is at least one of:
a) diffusion or permeation to, onto, into or through tissue or bodily fluid, including diffusion or permeation along concentration gradients;
b) mobility or permeation enhancement to, onto, into or through tissue or bodily fluid, including application of electroporation, sonoporation, osmotic mechanisms, tissue-heating which dilates microcapillaries or blood lumens, drugs, mechanical tissue-disruptors, including microneedle arrays, all of which increase permeability;
c) provision of an applied or assistive diffusion or permeation driving force to, onto, into or through tissue or body fluid, including acoustic streaming or application of electric fields as for electrophoretic or iontophoretic enhanced transport, all of which drive species; or
d) any transport in the bloodstream or in any other flowable or liquid bodily fluid.

28. The method of claim 26 wherein delivery to the first site includes any one or more of:
a) contacting the apparatus to a patient's bodily tissue or fluid for a short or prolonged period;
b) presenting the apparatus to a bodily tissue or fluid at a standoff distance and spraying, pouring, ejecting, extruding or dripping at least the species upon or into the patient's body tissue or fluid;
c) coupling the apparatus to a bodily tissue or fluid with a flowable or injection lumen which allows at least the species to be flow-delivered from the reservoir to, upon or into the patient's bodily tissue or fluid in any of a liquid, vaporous or gaseous form;
d) use of the apparatus to physically place on or in the body at least some preformed, metered or controlled amount or volume of at least the species in a solid or substantially unflowable form;
e) inhalation, ingestion or any type of bodily or limb immersion; or
f) the use of tissue disruption or disruptors, including a syringe or microneedle(s).

29. The method of claim 26 wherein the concentration of the species in the reservoir is higher than any one or more of:
a) the concentration that is found in an overall normal healthy body not utilizing the apparatus; or
b) the concentration that is found in an overall human body in comparison to the concentration attained by hyperbaric exposure methods.
